(19) United States
(12) Patent Application Publication
(10) Pub. No.: US 2005/0071911 A1
(43) Pub. Date: Apr. 7, 2005

(54) Mask for skin diving

(76) Inventor: Hsing-Chi Hsieh, Taipei (TW)

Correspondence Address: PRO-TECHTOR INTERNATIONAL, 20775 Norada Court, Saratoga, CA, 95070-3018, US

(21) Appl. No.: 10/680,627

(22) Filed: Oct. 6, 2003

(51) Int. Cl. A61F 9/02

(52) U.S. Cl. 2/428

(57) ABSTRACT

A mask for skin diving includes a skirt and two lenses laterally symmetrically mounted within the skirt. The skirt has two outer ends that are rearward inclined by a predetermined angle, and the lenses also have two outer ends that are rearward inclined by an angle corresponding to the skirt, so that a user's sight projecting toward two lateral sides could be widened without being blocked by the skirt. The angle by which the outer ends of the skirt and the lenses are rearward inclined is preferably within the range from 5 to 10 degrees.

### FIELD OF THE INVENTION

The present invention relates to a mask for skin diving, and more particularly to a mask for skin diving having two lenses that are rearward inclined at two outer ends to increase a user's viewing field.

### BACKGROUND OF THE INVENTION

A mask for skin diving typically includes a skirt and two lenses separately mounted at left and right positions within the skirt.

Conventionally, the two lenses mounted in the skirt of the mask are located within a straight line. That is, an angle contained between the two lenses is 180-degree. When a user wears the conventional mask during diving, two outer ends of the horizontally extended skirt would partially block the user's sight projected sideward. That is, the user's viewing field or viewing angle is undesirably narrowed. For people who must work under deep water, this is a confusing problem requiring improvement.

### SUMMARY OF THE INVENTION

A primary object of the present invention is to provide an improved mask for skin diving, so as to solve the problem of narrowed viewing field existed in the conventional mask having two in-line lenses.

To achieve the above and other objects, the mask for skin diving according to the present invention includes two lenses that are so located that two outer ends thereof are rearward inclined by a predetermined angle, and a skirt within which the two lenses are mounted also has two rearward inclined outer ends. With the two rearward inclined outer ends provided at the two lenses and the skirt, a user's sight projecting toward two lateral sides would not be blocked. That is, the user may have a widened viewing field during diving.

In a preferred embodiment of the present invention, the angle by which the skirt and the two lenses are rearward inclined at two outer ends is within the range from 5 to 10 degrees to allow the user to have a widened viewing field without a distorted image.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of a mask for skin diving according to the present invention; and

FIG. 2 is a fragmentary sectioned top view of the mask of FIG. 1.

### DETAILED DESCRIPTION OF THE PREFERRED EMBODIMENTS

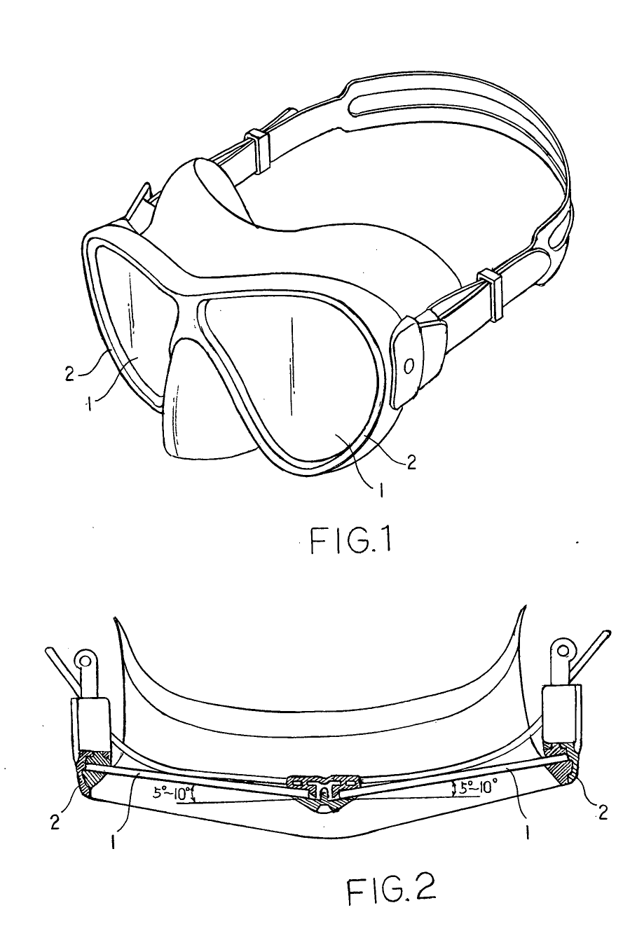

Please refer to FIGS. 1 and 2 that are perspective and sectioned top views, respectively, of a mask for skin diving according to a preferred embodiment of the present invention. As shown, the mask mainly includes two lenses 1 and a skirt 2. The two lenses 1 are laterally symmetrically mounted within the skirt 2. The two lenses 1 are so arranged that two outer ends thereof are rearward inclined by a predetermined angle. Meanwhile, the skirt 2 also has two outer ends that are rearward inclined corresponding to the inclined lenses 1.

When a user wears the mask of the present invention during skin diving, the skirt 1 with two rearward inclined outer ends would not block the user's sight projecting toward two lateral sides, and the two lenses 1 rearward inclined at two outer ends allows the user to have a laterally widened viewing field.

In a most preferred embodiment of the present invention, the angle by which the outer ends of the lenses 2 and the skirt 2 are rearward inclined is within the range from 5 to 10 degrees. A rearward inclination at the outer ends of the lenses 1 and the skirt 2 within this range allows the user to have a widened viewing field without a distorted image.

In brief, the present invention provides a mask for skin diving that is characterized in that two outer ends of the skirt and the two lenses thereof are rearward inclined by a predetermined angle to allow the user to have widened and non-distorted view field under water.

1. A mask for skin diving, comprising a skirt and two lenses laterally symmetrically mounted within said skirt; said skirt having two outer ends that are rearward inclined by a predetermined angle, and said lenses also having two outer ends that are rearward inclined by an angle corresponding to said skirt.

2. The mask for skin diving as claimed in claim 1, wherein said predetermined angle by which outer ends of said skirt and said lenses are rearward inclined is within the range from 5 to 10 degrees.
